(19) United States
(12) Patent Application Publication
(10) Pub. No.: US 2005/0169942 A1
(43) Pub. Date: Aug. 4, 2005

(54) Novel DNA sequences of the botulinum neurotoxin complex of Clostridium botulinum type A-Hall (Allergan) strain for production of therapeutics

(75) Inventors: Shengwen Li, Irvine, CA (US); Li Zhang, Thousand Oaks, CA (US); Kei Roger Aoki, Coto De Caza, CA (US); Wei-Jen Lin, Cerritos, CA (US)

Correspondence Address: Stephen Donovan;Allergan, Inc., 2525 Dupont Drive, Irvine, CA, 92612, US

(73) Assignee: ALLERGAN, INC., Irvine, CA (US)

(21) Appl. No.: 10/961,935

(22) Filed: Oct. 7, 2004

Related U.S. Application Data
(60) Provisional application No. 60/509,715, filed on Oct. 7, 2003.

(51) Int. Cl. A61K 39/08; C07H 21/04; C07K 14/195; C12N 1/21; C12N 15/74

(52) U.S. Cl. 424/239.1; 435/69.3; 435/252.3; 435/320.1; 530/350; 536/23.7

(57) ABSTRACT

This invention broadly relates to recombinant DNA technology, molecular biology, neuroscience, and medicine. Particularly, the present invention features novel sequences of the toxin and non-toxin components of the Clostridium botulinum toxin type A-Hall (Allergan) strain complex as well as the expression vector system in a heterologous organism and methods to express such nucleic acid sequences.

### PRIORITY

This application claims priority to provisional patent application Ser. No. 60/509,715 (Attorney Docket Number ALLE0009-001 (17639), filed Oct. 7, 2003), the disclosure of which is incorporated in its entirety herein by reference.

### FIELD OF INVENTION

This invention broadly relates to recombinant DNA technology, molecular biology, neuroscience, and medicine. Particularly, the present invention features novel sequences of the toxin and non-toxin components of the Clostridium botulinum toxin type A-Hall (Allergan or “AGN”) strain complex as well as the expression vector system in a heterologous organism and methods to express such nucleic acid sequence compositions for therapeutic applications.

### BACKGROUND

In 1885, Claude Bernard stated that “Poisons can be employed as a means for the destruction of life or as an agent for the treatment of the sick.” (Bernard, 1927). A century later, Dr. Alan B. Scott pioneered the preclinical evaluation of the purified native botulinum neurotoxin type A toxin complex from type A-Hall (Allergan or “AGN”) strain in monkeys and the subsequent clinical trials for the treatment of patients who suffer from the involuntary muscle disorders strabismus (wandering eye), blepharospasm, and hemifacial spasm in the early 1980s (Johnson, 1999). In 1989, the US Food and Drug Administration (FDA) licensed the purified native type A-Hall(AGN) toxin complex (manufactured as BOTOX®) for the treatment of these diseases caused by involuntary muscle contractions, particularly focal and segmental muscle movements (Schantz and Johnson, 1997). Since then, other clinical applications of BoNT/A-Hall (AGN) complex (BOTOX®) have been extended to include dozens of pathological conditions, characterized by spasm or overactivity of a particular muscle or group of muscles (Binder et al., 1998; Schnider et al., 1999; Binder et al., 2002). In many of these illnesses, the muscular hyperactivity is the primary disorder (e.g., cervical dystonia), while in others, it is secondary to a primary disease (e.g., rigidity and tremor in Parkinson's disease). Intramuscular injection of the purified native toxin complex from type A-Hall (AGN) strain in these disorders has replaced previous less satisfactory surgical or pharmacological treatments. The therapeutic effect of BoNT/A-Hall (AGN) typically lasts three to four months and, depending on the muscle type, it can last as long as 12 months (Brin and Jankovic, 2002).

Different strains of Clostridium botulinum produce structurally similar but immunologically distinct serotypes of BONT, including thus far seven characterized serotypes A, B, Cl, D, E, F, G (Henderson et al., 1997). All seven serotypes of BoNTs function as Zinc-dependent metalloproteases that inhibit the release of neurotransmitter acetylcholine from peripheral cholinergic synapses (Schiavo et al., 2000). These toxins, however, differ in their complex size, post-translational activation level (‘nicking’), substrate cleavage sites, receptor binding, muscle weakening efficacy, duration of action, and target affinity (Black and Dolly, 1986; Schiavo and Montecucco, 1997; Brin et al., 1999; Simpson, 2000; Aoki and Guyer, 2001).

The bacterium Clostridium botulinum type A has been used widely for the production of BoNT/A for studies such as neurotoxin biochemistry, pharmacology and crystallography (Montecucco et al., 1996; Lacy et al., 1998), and in the manufacture of the therapeutic agent BOTOX® (Manufatured with the purified native 900-kDa neurotoxin complex from the type A-Hall (AGN) strain: Aoki, 2001b; Aoki and Guyer, 2001). The progenitor BoNT/A produced by the type A-Hall (AGN) strain is a 900-kDa complex consisting of a highly activated (nicked) neurotoxin, a number of haemagglutinin (HA) molecules, botR, and a non-toxic non-hemagglutinin protein (NTNH) (Henderson et al., 1997). Although botR is well established as a transcription factor, little is known about the function of NTNH and HAs. NTNH and HAs may function as a chaperon for BONT trafficking. The complete nucleotide gene sequence of BoNT/A complex was previously determined from C. botulinum type A-NCTC 2916 strain (Thompson et al., 1990).

Despite the widespread medical applications of the purified native toxin complex of the type A-Hall (AGN) strain in human pathological conditions, its nucleotide sequence has not been determined. Here, we present the first report of the complete nucleotide sequence of the BoNT/A progenitor toxin complex of C.botulinum type A-Hall (AGN) strain. The DNA and the corresponding deduced amino acid sequences were compared to existing sequences of various neurotoxin serotypes deposited in GenBank. The sequence information presented here will provide a molecular basis for understanding the interaction between the toxin and nontoxic proteins in the complex, and will facilitate the investigation and characterization of toxic and nontoxic protein trafficking both in vitro and in vivo.

### SUMMARY OF THE INVENTION

The present invention features novel sequences of the medically applied toxin and non-toxin components of the Clostridium botulinum toxin type A-Hall (Allergan-AGN) strain complex. In some embodiments, the invention features an isolated nucleic acid molecule comprising a nucleotide sequence that encodes a botulinum toxin of type A-Hall (AGN) strain. In some embodiments, the invention also features isolated nucleic acid molecules comprising nucleotide sequences that encode novel non-toxic components of the Clostridium botulinum toxin type A-Hall (AGN) strain complex.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### DEFINITIONS

“BoNT” means botulinum neurotoxin.

“botR/OrfX” means botulinum regulatory protein/open reading frame X.

“HA” means hemagglutinin.

“NTNH” means non-toxic non-hemagglutinin.

“ORF” means open reading frame.

“PCR” means polymerase chain reaction.

“Promoter” means a DNA sequence at the 5′-end of a structural gene that is capable of initiating transcription.

“Operably linked” means two sequences of a nucleic acid molecule which are linked to each other in a manner which either permits both sequences to be transcribed onto the same RNA transcript, or permits an RNA transcript, begun in one sequence, to be extended into the second sequence. Thus, two sequences, such as a promoter and any other “second” sequence of DNA (or RNA) are operably linked if transcription commencing in the promoter sequence will produce an RNA (or cDNA) transcript of the operably linked second sequence. In order to be “operably linked” it is not necessary that two sequences be immediately adjacent to one another.

“Vector” means a nucleic acid sequence used as a vehicle for cloning or expressing a fragment of a foreign nucleic acid sequence. And a “vector operably harboring a nucleic acid sequence” means a vector comprising the nucleic acid sequence and is capable of expressing such nucleic acid sequence.

“Host” or “host cell” means the cell in which a vector is transformed. Once the foreign DNA is incorporated into the host cell, the host cell may express the foreign DNA. For example, the “host cell” of the present invention include Sf9, a clonal isolate of the IPLB-Sf21-AE line established from Spodoptera frugiperda, commonly known as the fall army worm.

“Light chain” (L chain, LC, or L) has a molecular weight of about 50 kDa. A light chain has proteolytic/toxic activity.

“Heavy chain” (H chain or H) has a molecular weight of about 100 kDa. A heavy chain comprises an H_{C} and an H_{N}.

“H_{C}” is the carboxyl end fragment of the H chain, which is involved in binding to cell surfaces possibly via a toxin receptor.

“H_{N}” is the amino end segment of the H chain, which is involved in the translocation of at least the L chain across an intracellular endosomal membrane into a cytoplasm of a cell.

“About” means approximately or nearly and in the context of a numerical value or range set forth herein means ±10% of the numerical value or range recited or claimed.

### BRIEF DESCRIPTION OF THE DRAWINGS

FIG. 1. Strategy for cloning neurotoxin complex genes of the type A-Hall (AGN) strain of C. botulinum. The top solid line represents the genomic DNA. The open rectangles represent the open reading frame of the particular genes. The positions of genes encoding the components of the BoNT/A complex are arranged according to the neurotoxin cluster on the chromosome of C.botulinum type A-NCTC 2916 strain. Arrows indicate the position of the primers used to amplify the gene fragments. The primer name is next to the arrow (5′-end is the forward sense primer While 3′-end is the backward anti-sense primer).

The pBoNT (LC+HC) represents for the cloning recombinant plasmid for encoding botulinal neurotoxin light chain and heavy chain; pNTNH is for nontoxic nonhemagglutinin; pHA70, pHA17, pHA34 are for hemagglutinin components HA70, HA17, and HA34, respectively; and pbotR/OrfX is for a putative regulatory protein X.

FIG. 2. Nucleotide and amino acid sequences of the genes for botulinum toxin complex of Clostridium botulinum type A-Hall (AGN) strain (SEQ ID NO: 1, SEQ ID NO: 2). The illustrated sequence was derived from the inserts of two independently PCR-generated, duplicated clones of recombinant plasmids. The encoded amino acids are in single-letter code below the first nucleotide of the corresponding codon.

For BONT: Three domains are indicated: LC, light chain, the catalytic domain, a.a. residue 1-437; H_{N}, heavy chain N-terminal, the transmembrane domain, a.a. residue 449-872; H_{C}, heavy chain C-terminal, the receptor binding domain, a.a. residue 873-1296. Nicking peptide (underlined with a square dot line): Residues 437 to 448, the majority of which are cleaved during post-translational modification, are adopted from the conserved sequence obtained by DNA sequences of strains NCTC2916 and 62A. As circled letter, two cysteine residues (conserved Cys-430, LC; Cys-454, HC) are involved in disulfide bond formation between the L- and H-chains. Boxed is the histidine-rich motif between positions 223-230 (HELxHxxH) associated with metalloprotease activity, the conserved zinc-binding motif (Bold letters are consensus). Underlined with a solid line is a PYxGxAL motif (BoNT/A positions 635-645), located adjacent to a region identified as membrane spanning. Oval circled is a di-leucine motif (residues 427-428 of LC) present in type A toxin gene, which may be critical for toxin trafficking.

FIG. 3. Alignment of BoNT/A-Hall (AGN) with the known protein sequences of other serotypes. A: Scheme of BoNT protein structure of Clostridium botulinum type A Hall (AGN)-strain. The three domains of BoNT/A are indicated: LC, light chain, the catalytic domain, a.a. residue 1-437; H_{N}, heavy chain N-terminal, the transmembrane domain, a.a. residue 449-872; H_{C}, heavy chain C-terminal, the receptor binding domain, a.a. residue 873-1296. Nicking peptide: Residues 437 to 448, the majority of which are cleaved during post-translational modification, are adopted from the conserved sequence obtained by DNA sequences of strains NCTC2916 and 62A.

The BoNT of type A-Hall (AGN) strain has conserved regions as follows: 1) Two cysteine residues (conserved Cys-430, LC; Cys-454, HC), which are involved in disulfide bond formation between the L and H chains; 2) a histidine-rich motif between positions 223-230 (HELxHxxH) associated with metalloprotease activity; and 3) a PYxGxAL motif (BoNT/A positions 635-645), located adjacent to a region identified as membrane spanning. Consistent with previous findings, however, the C-terminal portion of the BoNT/A-Hall-(AGN)-HC shows comparatively high sequence differences. There is a di-leucine motif (residues 427-428 of LC) only present in type A toxin gene, which may be critical for toxin trafficking. B: BoNT/A-Hall (AGN) contains several potential sites for phosphorylation by casein kinase II (*), protein kinase C (#), tyrosine kinases (@), glycogen synthase kinase 3 (&), cGMP dependent protein kinase (PKG) (%) that are well conserved. Note that BoNT/A-Hall (AGN) also contains well conserved N-glycosylation sites ($).

Alignment display setup is as follows. Non-similar: black lettering, white background; Conservative: Dark blue lettering, light blue background; Block of similar: black lettering, green background; Identical: read lettering, yellow background.

FIG. 4. Phylogenetic dendrogram summarizing the compilation of the current available sequence data for the selective genus clostridium. A: BoNTs; B: NTNHs; C: HA70s.

FIG. 5. Nucleic acid sequence (SEQ ID NO: 3) and amino acid sequence (SEQ ID NO: 4) of Hall A/AGN NTNH.

FIG. 6. Nucleic acid sequence (SEQ ID NO: 5) and amino acid sequence (SEQ ID NO: 6) of Hall A/AGN HA70.

FIG. 7. Nucleic acid sequence (SEQ ID NO: 7) and amino acid sequence (SEQ ID NO: 8) of Hall A/AGN HA34.

FIG. 8. Nucleic acid sequence (SEQ ID NO: 9) and amino acid sequence (SEQ ID NO: 10) of Hall A/AGN HA17.

FIG. 9. Nucleic acid sequence (SEQ ID NO: 11) and amino acid sequence (SEQ ID NO: 12) of Hall A/AGN botR/OrfX.

### DESCRIPTION OF EMBODIMENTS

The present invention relates to novel sequences of the complex of Clostridium botulinum toxin type A-Hall (AGN) strain. The invention features an isolated nucleic acid molecule comprising a nucleotide sequence (SEQ ID NO: 1) that encodes a Hall A/AGN botulinum toxin. In some embodiments, the nucleotides at positions 3589, 3590 and 3591 are GCU, respectively. In some embodiments, the nucleotides at positions 3589, 3590 and 3591 are GCC, respectively. In some embodiments, the nucleotides at positions 3589, 3590 and 3591 are GCG, respectively. Without wishing to limit the invention to any theory or mechanism of operation, it is believed that the above referenced GCU, GCC or GCG may allow for the expression of a toxin that may complex with non-toxic components (e.g., described below) to form a 900 kDa complex.

The invention also features isolated nucleic acid molecules comprising nucleotide sequences that encode the non-toxic components of the Clostridium botulinum toxin type A-Hall (AGN) strain complex. For example, the present invention features an isolated nucleic acid molecule comprising a nucleotide sequence (SEQ ID NO: 3) that encodes a Hall A/AGN NTNH, a nucleotide sequence (SEQ ID NO: 5) that encodes a Hall A/AGN HA70, a nucleotide sequence (SEQ ID NO: 7) that encodes a Hall A/AGN HA34, a nucleotide sequence (SEQ ID NO: 9) that encodes a Hall A/AGN HA17, and/or a nucleotide sequence (SEQ ID NO: 11) that encodes a Hall A/AGN botR/OrfX.

The sequences present have been submitted to GenBank with Accession numbers: AF488749 (BoNT), AF488748 (NTNH), AF488747 (HA70), AF488746 (HA17), AF488745 (HA17), AF488750 (botR/OrfX), which are incorporated in their entirety by reference herein.

In some embodiments, the isolated nucleic acid molecule comprises a nucleotide sequence that is more than 95% homologous to the SEQ ID NO: 1, 3, 5, 7, 9, or 11. In some embodiments, these nucleotide sequences encode for amino acid sequence 2, 4, 6, 8,10 and 12, respectively. Percent homology can be determined by, for example, the Gap program (Wisconsin Sequence Analysis Package, Version 8 for Unix, Genetics Computer Group, University Research Park, Madision Wis.), which uses the algorithm of Smith and Waterman (Adv. Appl. Math., 1981, 2, 482-489, which is incorporated in its entirety herein by reference) using the default settings.

In some embodiments, the isolated nucleic acid molecule comprises a nucleotide sequence that is more than 96% homologous to the SEQ ID NO: 1, 3, 5, 7, 9, or 11. In some embodiments, these nucleotide sequences encode for amino acid sequence 2, 4, 6, 8, 10 and 12, respectively.

In some embodiments, the isolated nucleic acid molecule comprises a nucleotide sequence that is more than 97% homologous to the SEQ ID NO: 1, 3, 5, 7, 9, or 11. In some embodiments, these nucleotide sequences encode for amino acid sequence 2, 4, 6, 8, 10 and 12, respectively.

In some embodiments, the isolated nucleic acid molecule comprises a nucleotide sequence that is more than 98% homologous to the SEQ ID NO: 1, 3, 5, 7, 9, or 11. In some embodiments, these nucleotide sequences encode for amino acid sequence 2, 4, 6, 8, 10 and 12, respectively.

In some embodiments, the isolated nucleic acid molecule comprises a nucleotide sequence that is more than 99% homologous to the SEQ ID NO: 1, 3, 5, 7, 9, or 11. In some embodiments, these nucleotide sequences encode for amino acid sequence 2, 4, 6, 8, 10 and 12, respectively.

In some embodiments, the isolated nucleic acid molecule comprises a nucleotide sequence that is more than 99.20% homologous to the SEQ ID NO: 1, 3, 5, 7, 9, or 11. In some embodiments, these nucleotide sequences encode for amino acid sequence 2, 4, 6, 8, 10 and 12, respectively.

In some embodiments, the isolated nucleic acid molecule comprises a nucleotide sequence that is more than 99.40% homologous to the SEQ ID NO: 1, 3, 5, 7, 9, or 11. In some embodiments, these nucleotide sequences encode for amino acid sequence 2, 4, 6, 8, 10 and 12, respectively.

In some embodiments, the isolated nucleic acid molecule comprises a nucleotide sequence that is more than 99.60% homologous to the SEQ ID NO: 1, 3, 5, 7, 9, or 11. In some embodiments, these nucleotide sequences encode for amino acid sequence 2, 4, 6, 8, 10 and 12, respectively.

In some embodiments, the isolated nucleic acid molecule comprises a nucleotide sequence that is more than 99.80% homologous to the SEQ ID NO: 1, 3, 5, 7, 9, or 11. In some embodiments, these nucleotide sequences encode for amino acid sequence 2, 4, 6, 8, 10 and 12, respectively.

In some embodiments, the isolated nucleic acid molecule comprises a riucleotide sequence that is more than 99.90% homologous to the SEQ ID NO: 1, 3, 5, 7, 9, or 11. In some embodiments, these nucleotide sequences encode for amino acid sequence 2, 4, 6, 8, 10 and 12, respectively.

The present invention also features vectors that comprise the nucleic acid molecules described herein. In some embodiments, a vector used in accordance with this invention may be a viral-based expression vector. In some embodiments, a vector used in accordance with this invention may be a plasmid-based expression vector. The viral-based or plasmid-based expression vector may be a yeast expression vector, a bacterial expression vector, a plant expression vector, an amphibian expression vector or a mammalian expression vector. In some embodiments, the present invention also features a cell-free expression system, e.g., the Roche system (see below).

The present invention also features host cells that comprise the vectors described herein. The host cells may be prokaryotic or eukaryotic cells. Non-limiting examples of prokaryotic host cells include Escherichia coli cell, Clostridium botulinum cell, Clostridium tetani cell, Clostridium beratti cell, Clostridium butyricum cell, and Clostridium perfringens cell. Non-limiting examples of eukaryotic host cells include yeast cells, plant cells, amphibian cells, mammalian cells, and insect cells. Non-limiting examples of yeast cells include a Saccharomyces cerevisiae cell, Schizosaccharomyces pombe cell, Pichia pastoris cell, Hansenula polymorpha cell, Kluyveromyces lactis cell and Yarrowia lipolytica cell. Non-limiting example a mammalian cell includes CHO cells. Non-limiting examples of insect cell include a Spodoptera frugiperda cell (e.g., Mimic Sf9 and Sf21 Insect cell line), Aedes albopictus cell, Trichoplusia nicell (e.g., BTI-Tn-5B1-4 cell line), Estigmene acrea cell, Bombyx mori cell and Drosophila melanogaster cell. The present invention also features organisms comprising the vectors described herein. Non-limiting examples of organisms include an insect larvae.

The present invention also features cell-free expression system that comprises the construction of toxin gene and non-toxin genes into an E.coli-based pIVEX2.3d vector (Roche Applied Science, Indianapolis, Ind.). Such a construct, pIVEX2.3d-BoNT/A, can be applied in the Rapid Translation System (RTS) 100 E. coli HY kit or RTS 9000 E.coli HY kit for a large scale (Roche Applied Science, Indianapolis, Ind.). In some embodiments, the cell-free expression system is selected from the group consisting of a wheat extract, a rabbit reticulocyte extract and an E.coli extract.

In some embodiments, the invention also features expression vector systems for simultaneously expressing and assembling of all components of type A-Hall (AGN) toxin complex as therapeutics. For example, a baculovirus expression vector system may be used for co-infection of all the six component genes and assembly of the functional complex as the therapeutic agent. See U.S. patent application Ser. No. 10/715,810, the disclosure of which is incorporated in its entirety herein by reference.

The present invention also features compounds comprising an amino acid sequence of the non-toxic components of the complex of Clostridium botulinum toxin type A-Hall (AGN) strain. For example, in some embodiments, the present invention features a compound comprising an amino acid sequence (SEQ NO: 2) of a Hall A/AGN botulinum toxin, an amino acid sequence (SEQ NO: 4) of a Hall ANAGN NTNH; an amino acid sequence (SEQ NO: 6) of a Hall A/AGN HA70; an amino acid sequence (SEQ NO: 8) of a Hall A/AGN HA34, an amino acid sequence (SEQ NO: 10) of a Hall A/AGN HA17, an amino acid sequence (SEQ NO: 12) of a Hall A/AGN botR/OrfX.

In some embodiments, the compound comprises an amino acid sequence that is more than 95% homologous to the SEQ ID NO: 2, 4, 6, 8, 10 or 11. In some embodiments, the compound comprises an amino acid sequence that is more than 96% homologous to the SEQ ID NO: 2, 4, 6, 8, 10 or 11. In some embodiments, the compound comprises an amino acid sequence that is more than 97% homologous to the SEQ ID NO: 2, 4, 6, 8, 10 or 11. In some embodiments, the compound comprises an amino acid sequence that is more than 98% homologous to the SEQ ID NO: 2, 4, 6, 8, 10 or 11. In some embodiments, the compound comprises an amino acid sequence that is more than 99% homologous to the SEQ ID NO: 2, 4, 6, 8, 10 or 11.

In some embodiments, a nucleotide sequence or amino acid sequence of the present invention may be administered to a mammal for therapeutic purposes. Accordingly, a nucleotide sequence or amino acid sequence of the present invention may be admixed, encapsulated, conjugated or otherwise associated with other molecules or mixtures of compounds as, for example, liposomes, formulations (oral, rectal, topical, etc.) for assisting in uptake, distribution and/or absorption.

Pharmaceutical compounds, compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Preferred topical formulations include those in which the compounds of the invention are in admixture with a topical delivery agent such as lipids, liposomes, fatty acids, fatty acid esters, steroids, chelating agents and surfactants. Preferred lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA). Compounds of the invention may be encapsulated within liposomes or may form complexes thereto, in particular to cationic liposomes. Alternatively, compounds may be complexed to lipids, in particular to cationic lipids. Preferred fatty acids and esters include but are not limited arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C₁₋₁₀ alkyl ester (e.g. isopropylmyristate IPM), monoglyceride, diglyceride or pharmaceutically acceptable salt thereof. Topical formulations are described in detail in U.S. patent application Ser. No. 09/315,298 filed on May 20, 1999 which is incorporated herein by reference in its entirety.

Compounds, compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Preferred oral formulations are those in which compounds of the invention are administered in conjunction with one or more penetration enhancers surfactants and chelators. Preferred surfactants include fatty acids and/or esters or salts thereof, bile acids and/or salts thereof. Preferred bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate and sodium glycodihydrofusidate. Preferred fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g. sodium). Also preferred are combinations of penetration enhancers, for example, fatty acids/salts in combination with bile acids/salts. A particularly preferred combination is the sodium salt of lauric acid, capric acid and UDCA. Further penetration enhancers include polyoxyethylene-9-lauryl ether, polyoxyethylene-20-cetyl ether. Compounds of the invention may be delivered orally, in granular form including sprayed dried particles, or complexed to form micro or nanoparticles. Compound complexing agents include poly-amino acids; polyimines; polyacrylates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pollulans, celluloses and starches. Particularly preferred complexing agents include chitosan, N-trimethylchitosan, poly-L-lysine, polyhistidine, polyornithine, polyspermines, protamine, polyvinylpyridine, polythiodiethylamino-methylethylene P(TDAE), polyaminostyrene (e.g. p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG).

Compounds, compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Pharmaceutical compounds and compositions of the present invention include, but are not limited to, solutions, emulsions, and liposome-containing formulations. These compounds may be generated from a variety of components that include, but are not limited to, preformed liquids, self-emulsifying solids and self-emulsifying semisolids.

The pharmaceutical compositions and formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compounds of the present invention may be formulated into any of many possible dosage forms such as, but not limited to, tablets, capsules, gel capsules, liquid syrups, soft gels, suppositories, and enemas. The compounds of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

In one embodiment of the present invention the pharmaceutical compounds may be formulated and used as foams. Pharmaceutical foams include formulations such as, but not limited to, emulsions, microemulsions, creams, jellies and liposomes. While basically similar in nature these formulations vary in the components and the consistency of the final product. The preparation of such compounds and formulations is generally known to those skilled in the pharmaceutical and formulation arts and may be applied to the formulation of the compounds of the present invention.

The sequencing of the novel sequences of the toxin and non-toxin components of the Clostridium botulinum toxin type A-Hall (AGN) strain complex herein were performed as follows:

1. Materials and Methods

1.1. Bacterial Growth and Chromosomal DNA Purification

C. botulinum type A-Hall (Allergan) strain (simplified as Hall-A strain or Hall-A-AGN) was originally provided by E. Schantz from the Food Research Institute at the University of Wisconsin-Madison. The bacterium was grown in the brain heart infusion (BHI) medium (DIFCO Laboratories, Detroit, Mich.) at 37° C. in an anaerobic jar with an anaerobic envelope (BBL). Chromosomal DNA was purified as previously described (Lin and Johnson, 1991). DNA concentration was determined by measuring the absorbency at 260/280 nm using spectrophotometry.

1.2. Amplification of Neurotoxin Complex Genes by Polymerase Chain Reaction (PCR) and Cloning of PCR-Amplified Fragments

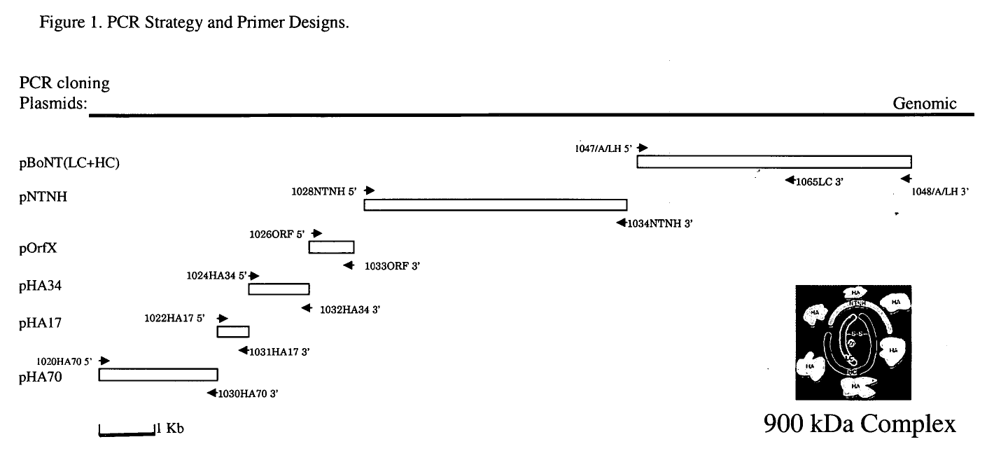

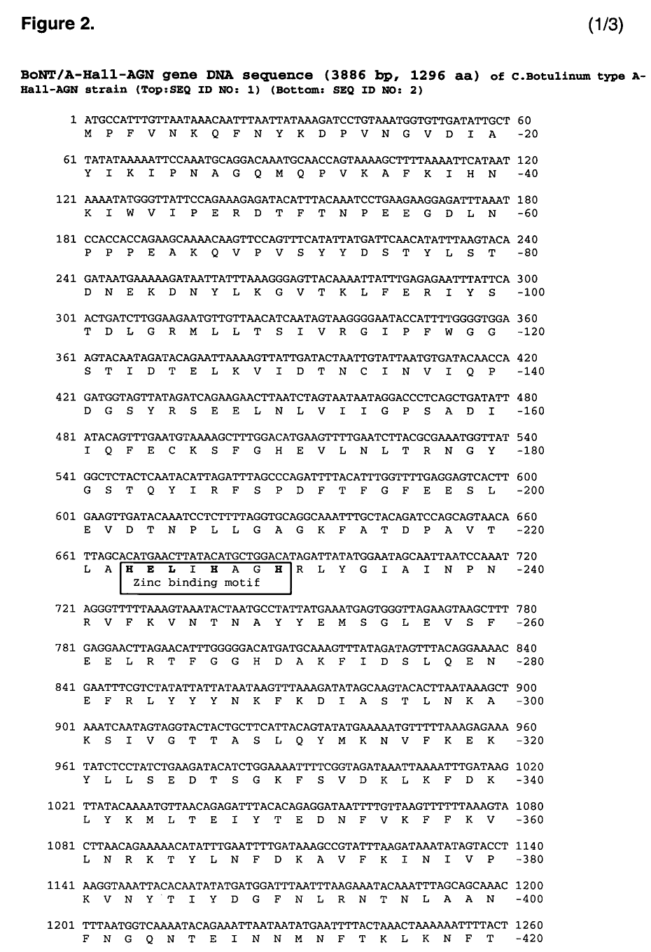

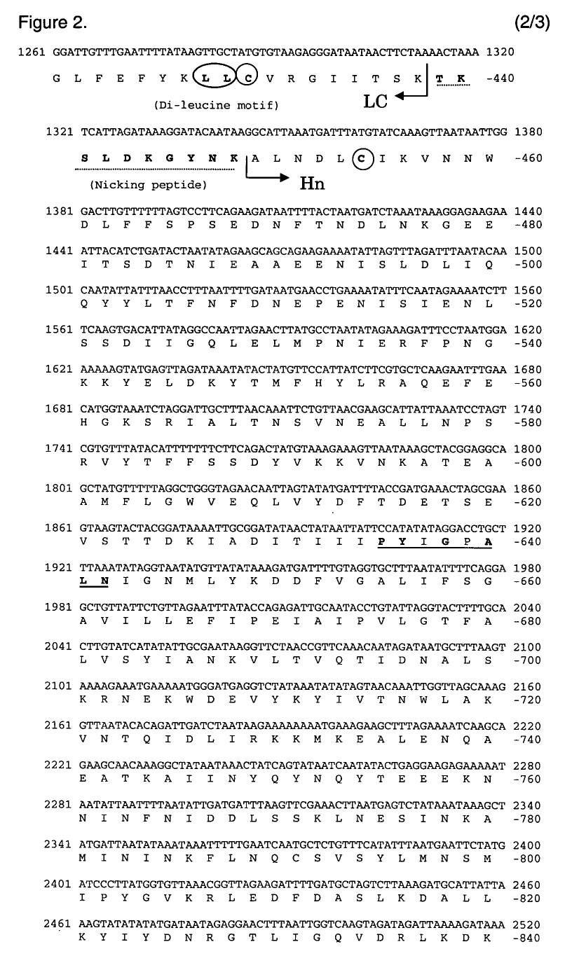

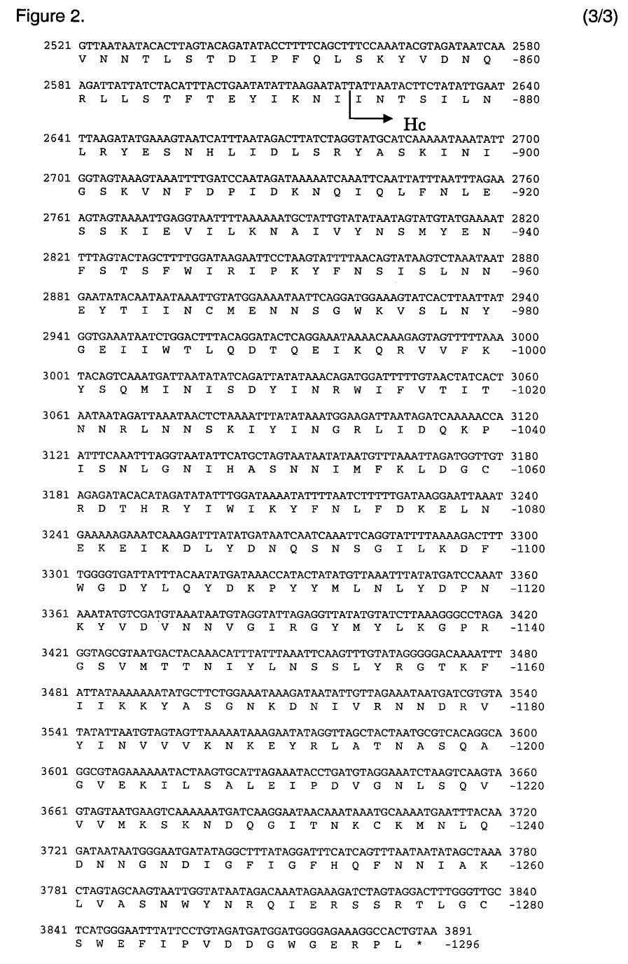

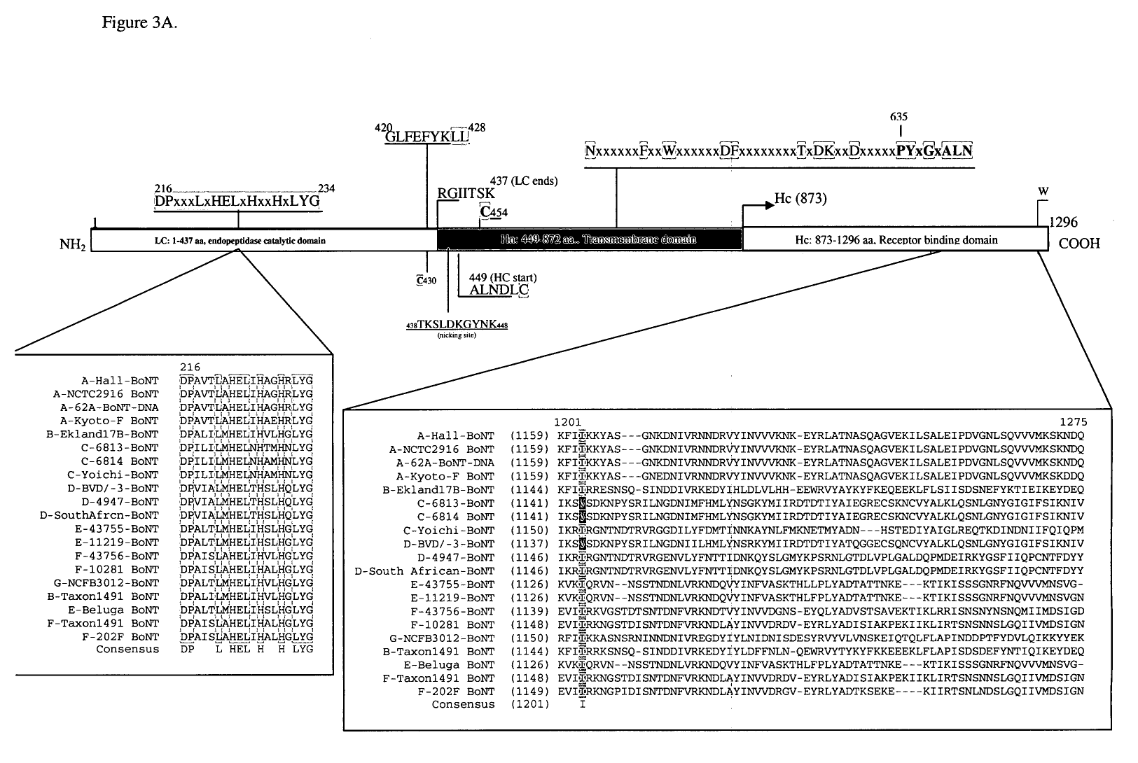

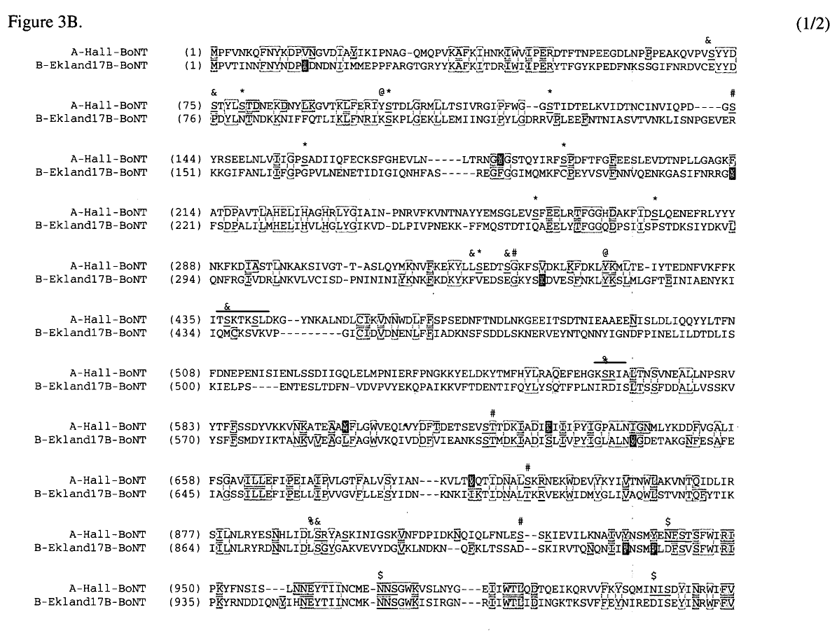

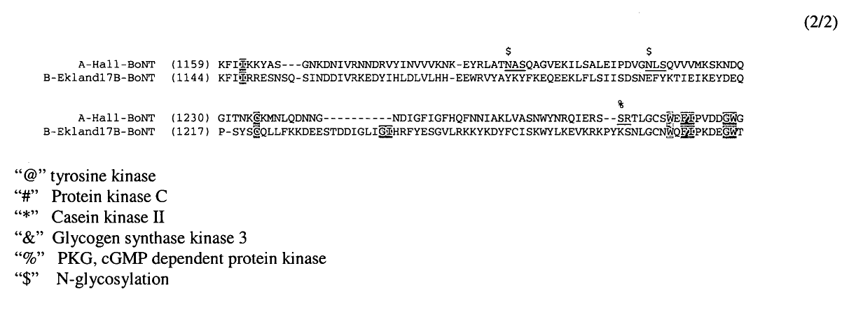

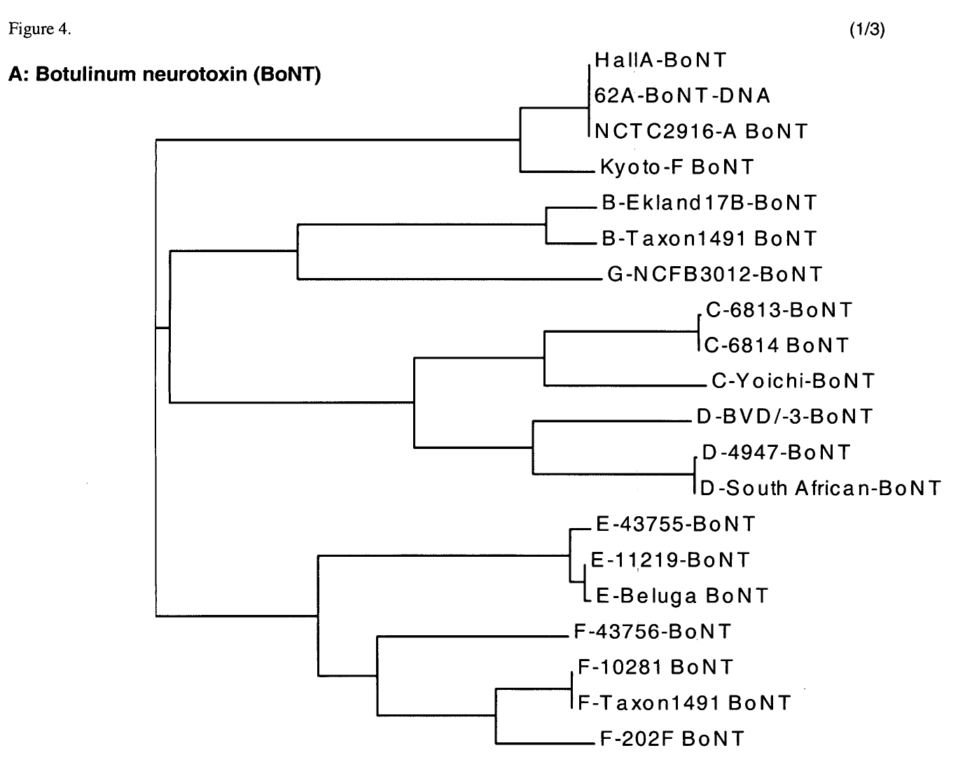

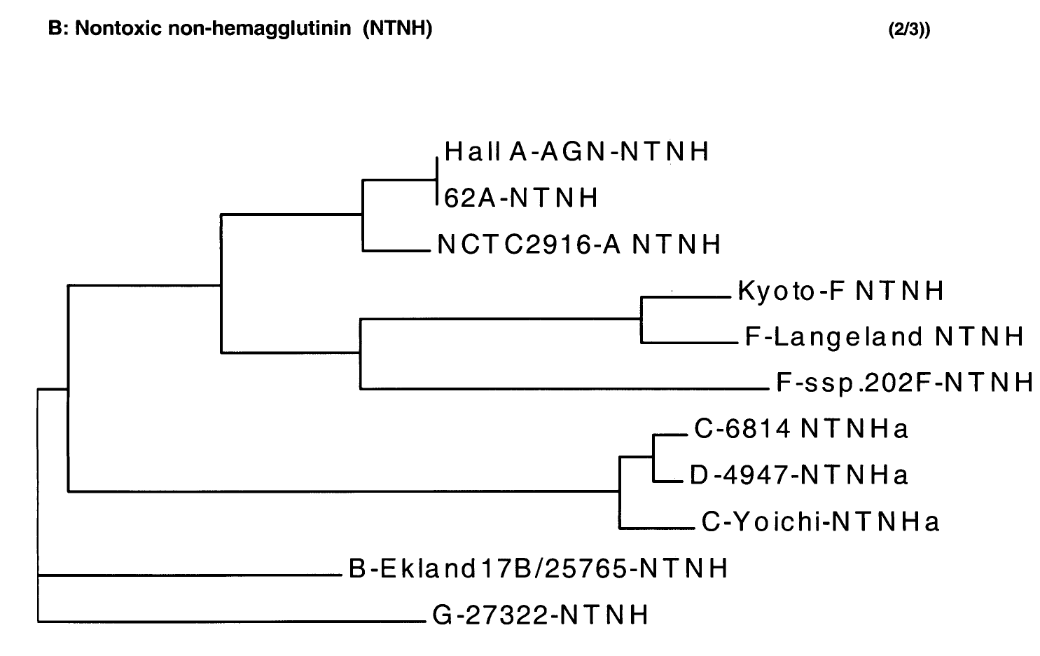

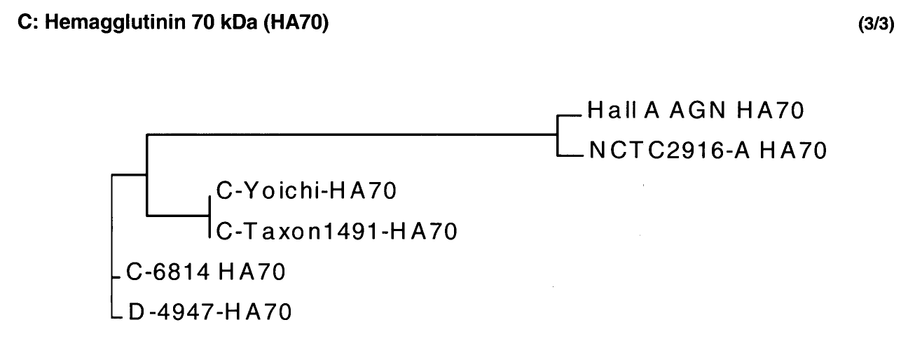

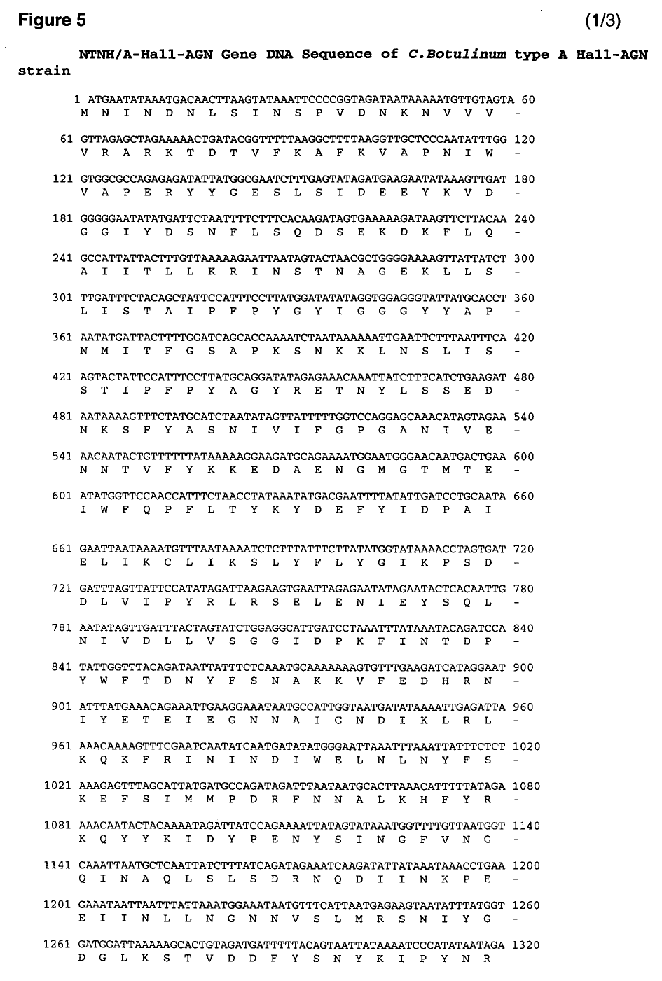

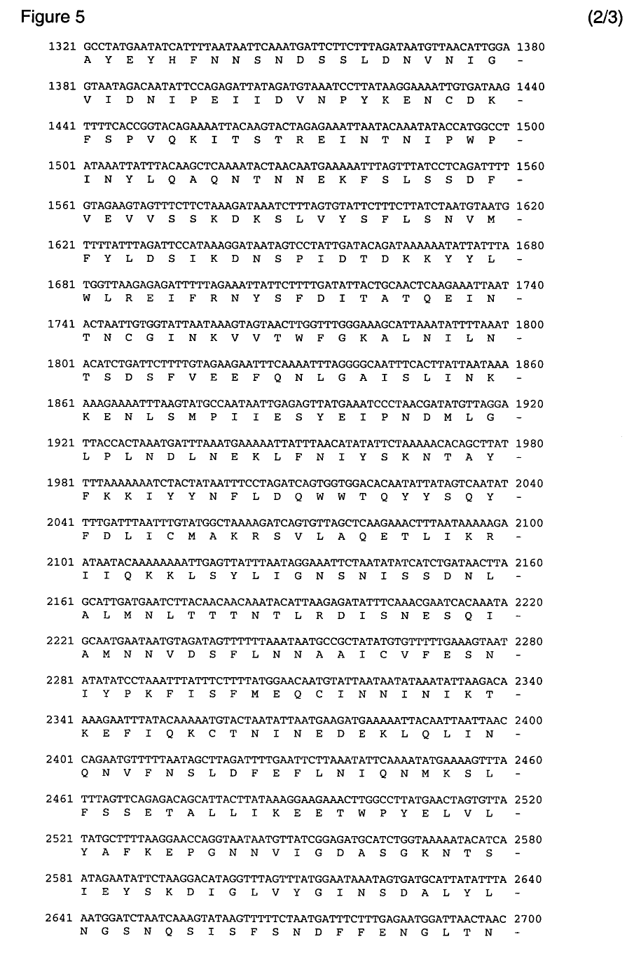

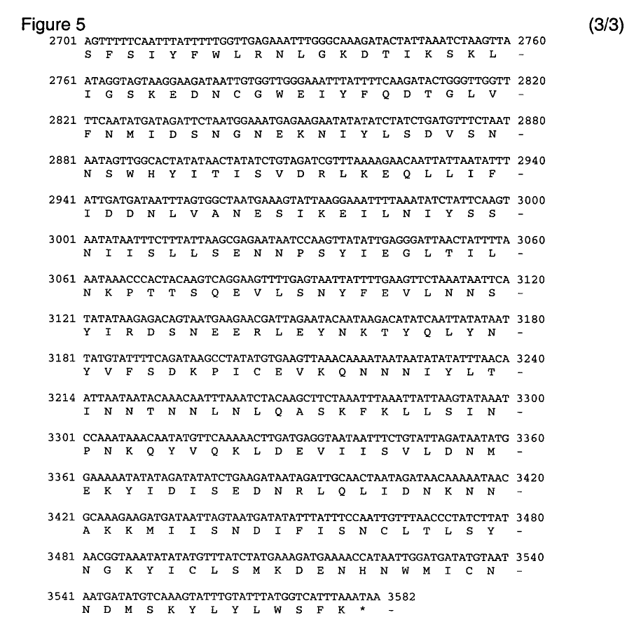

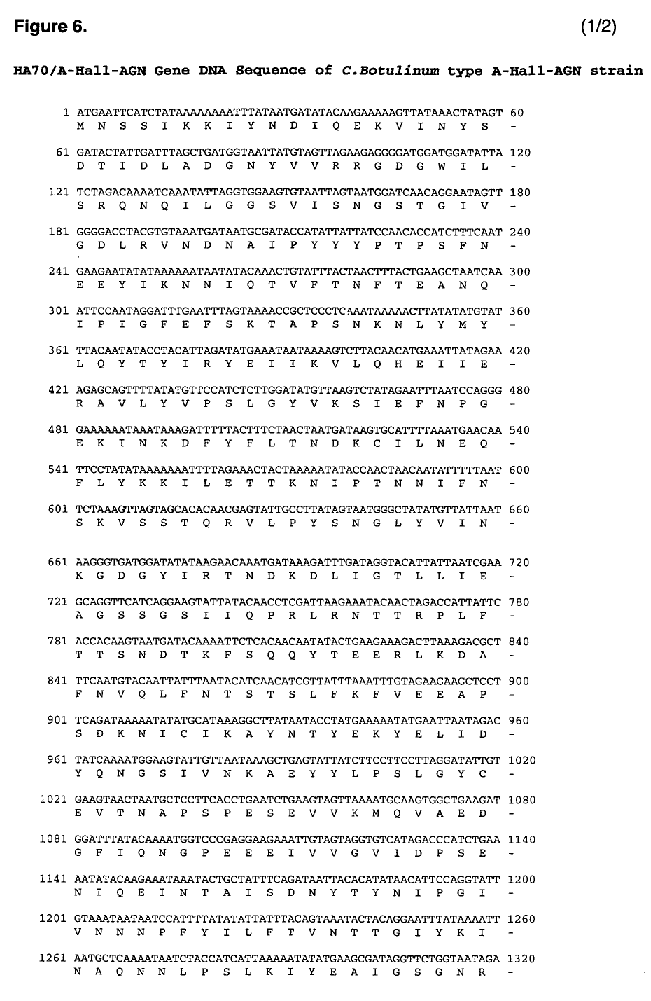

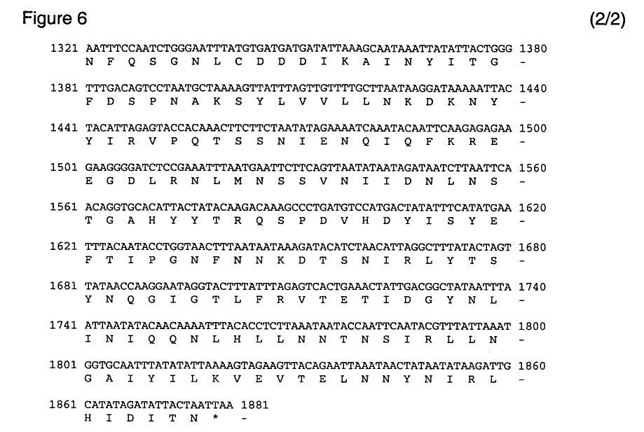

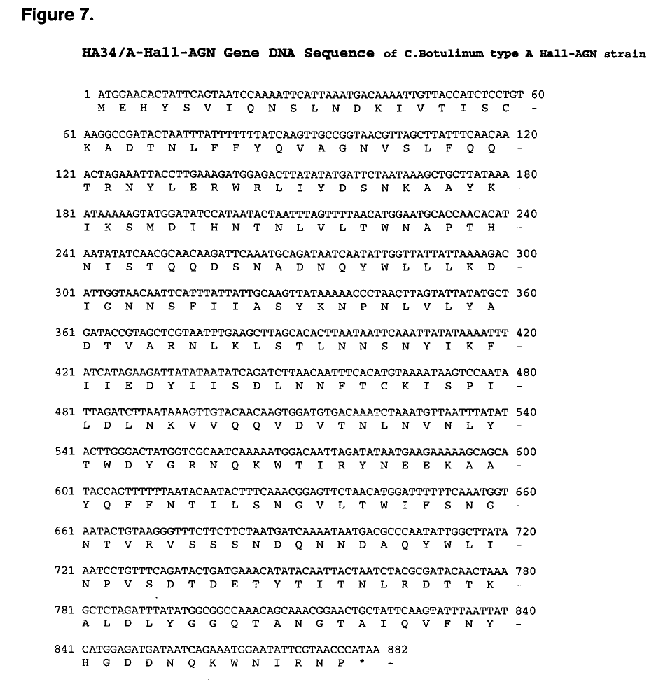

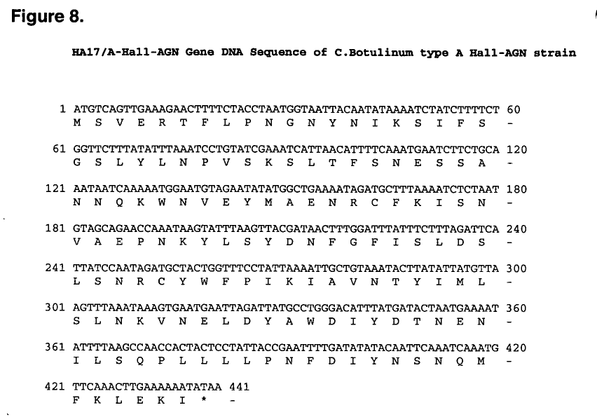

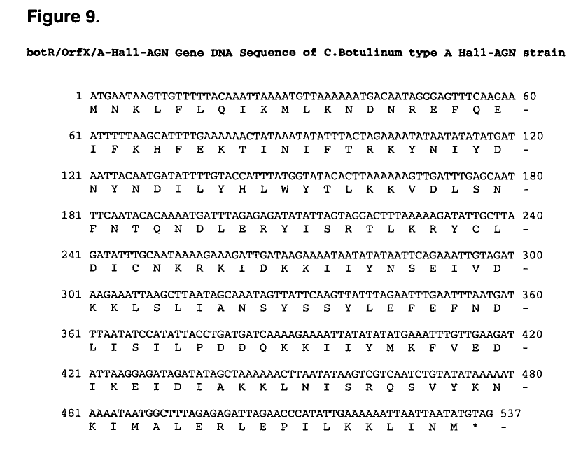

Each of the 6 open reading frames (ORFs) in the BoNT/A toxin gene cassette was first amplified by polymerase chain reaction (FIG. 1). PCR primers were designed on the conserved regions of published gene sequences of C. botulium type A-NCTC2916 strain (Thompson et al., 1990) and are listed in Table 1 and FIG. 1, which were synthesized by Sigma Genosys (Woodlands, Tex.). For subcloning convenience, a BamHI restriction sequence was engineered into the 5′-end of forward PCR primers, while a SacI or PstI recognition sequences were engineered into the 3′-end reverse PCR primers for nontoxic genes and the neurotoxin gene, respectively. PCR reactions were performed using the Expand™ High Fidelity PCR system (Boehringer-Mannheim, Indianapolis, Ind.) on the GeneAmp PCR System 9700 (PE Applied Biosystems, Foster City, Calif.). For each PCR, 1 ug of genomic DNA from Hall A strain was used as the template. Conditions for amplification 5were as follows: 96° C. for 2 min; followed by 25 cycles of 96° C. for 45 sec, 55° C. for 1 min, and 72° C. for 2 or 3 min (depending on the fragment size); 72° C. for 7 min (final extension). For PCR amplification of the longest fragments such as BoNT/A and NTNH genes, the GeneAmp XL PCR Kit (PE Applied Biosystems) was used according to the manufacturer's instructions.

The expected sizes of the PCR products for HA70, HA17, HA34, and botR, respectively, were then desalted by gel purification and concentrated using Microcon® YM-30 centrifugal filter device (Millipore, Bedford, Mass.) and then cloned into vector pCR/Blunt using the Zero Blunt PCR Cloning Kit (Invitrogen Corp, Carlsbad, Calif.). However, the PCR products with the correct size for BoNT/A and NTNH genes were gel-purified and cloned into the vector pCR2.1 for its larger capacity with TA Cloning Kit (Invitrogen Corp, Carlsbad, Calif.). For further subcloning in expression, correct insert orientation was first confirmed by restriction digestion. Two independent PCR reactions were performed for each ORF and two clones were isolated from each PCR reaction. Therefore, a total of 4 clones were reserved for each ORF for further DNA sequencing analyses.

1.3. DNA Sequencing and Analysis of the Deduced Amino Acid Sequences

Plasmid DNAs were purified using QIAGEN miniprep kit (QIAGEN, Valencia, Calif.) and the cloned inserts were sequenced utilizing the ABI Prism 377 DNA Sequencing System (Sequetech Corp., Mountain view, Calif.). M13 forward and reverse primers were used as the external primers and internal primers were synthesized subsequently to complete the sequencing. Table 1 PCR primers were used. All of the 4 clones from 2 independent PCR reactions were sequenced from 5′- and 3′-end directions. DNA and deduced amino acid sequences were analyzed with the analyzing tools at the National Center of Biotechnology Information (NCBI), the GCG program from Genetic Computing Group (Madison, Wis.; In-house Allergan Bioinformatics) and the Vector NTI suite from InforMax (Rockville, Md.).

2. Results and Discussion

2.1. Generation of Neurotoxin Gene and Nontoxin Genes by PCR

Approximately 40 kilobases (KB) of genomic DNA of type A-Hall (AGN) strain were purified and determined by agarose gel electrophoresis and this preparation was used as the DNA template in all PCR reactions for the amplification of nontoxin genes ntnh, ha70, ha34, ha17, botR and bonttoxin gene.

Primers designed to the conserved regions of the C. botulinum/A-NCTC 2916 strain were used to amplify neurotoxin and nontoxin genes using the template of C. botulinum type A-Hall (AGN) strain genomic DNA by PCR (Refer to Table 1 and FIG. 1). All six open reading frames encoding the BONT and nontoxic proteins (NTNH, HA70, HA34, HA17) and the regulatory protein (botR) were sequenced. To confirm the nucleotide sequences, we sequenced four clones: two clones for each PCR product and two independent PCR products for each gene.

2.2. DNA Sequence Analysis and Characterization of Deduced Protein Sequences

Detailed information on DNA and their deduced amino acid sequence analysis from these six ORFs encoding the BONT and nontoxic proteins of C. botulinum type A-Hall (AGN) strain are shown in Table 2. The bont gene encodes a protein of 1296 amino acid with a calculated molecular weight of 149.4 kDa. The hemagglutinin genes ha34, ha17, ha70, encode proteins of 291 a.a. (33.826 kDa), 147 a.a. (17.035 kDa), and 625 a.a. (71.144 kDa), respectively. The nontoxic non-hemagglutinin gene, ntnh, encodes a protein of 1193 a.a. of 138.218 kDa. The regulatory gene, botR, encodes a protein of 178 amino acids with 21.654 kDa in molecular weight.

Previous studies indicate that, regardless of origin, all the determined neurotoxin nucleotide sequences exhibit a codon usage characteristic of clostridial genes: codons ending in A or T are generally preferred. This codon bias reflects the low G+C content of the genes (24.51-27.84). The genes encoding for the neurotoxin complex of C. botulinum A-Hall (AGN) strain total 11,475 bp in length and have a GC content of 25.21%.

2.3. Comparison of Amino Acid Sequences of BONT Complex Between Type A-Hall (AGN) Strain and Other Clostridial Serotypes

Table 3 shows the comparative amino acid sequence identity and homology of the neurotoxin complex proteins between C. botulinum type A-Hall (AGN) strain and the other strains of C. botulinum with known DNA sequences. Overall, an amino acid sequence from the type A-Hall (AGN) strain shows the highest degree of identity with other type A strains. However, various BONT serotypes (A, B, C, D, E, F, G) exhibit a considerable degree of amino acid sequence heterogeneity when compared to the sequences in Type A-Hall (AGN) strain (˜60% heterogeneity and ˜40% homology). Comparative alignments of the amino acid sequences of BoNT/A show a 98˜100% sequence identity among different strains of A serotypes, except for Kyoto-F (90%), whereas the sequence identity between BoNT/A-Hall (AGN) and other toxin serotypes is only 30.4˜39.1%.

Similar to the neurotoxin, the toxin-associated proteins and the regulatory protein BotR from the type A-Hall (AGN) strain share more than 95% identity to the homologous proteins found in NCTC2916/A. Among all of the toxin associated proteins, NTNHs and HA70s are the most conserved, with 65˜87% identity across different serotypes. On the other hand, HA34s, present only in serotypes A-D, show greater diversity than all other toxin-associated proteins. HA34/A has ˜90% identity to HA34/B and only ˜35% identity to HA34/C and HA34/D. Relatively higher sequence identity (˜60%) is seen with HA17 and BotR of the type A-Hall (AGN) strain compared to their respective counterparts in serotypes C or D. Of all proteins within the toxin complex, the highest degree of conservation of NTNH and HA70 across different serotypes may underscore a critical role for these proteins in the formation of toxin complexes.

Comparative alignment indicates that the BoNT/A-Hall (AGN) strain contains some common domains or motifs although it is highly different from other serotypes (FIG. 3A). The BONT of type A-Hall (AGN)strain has conserved regions as follows: 1) Two cysteine residues (conserved Cys-430, LC; Cys-454, HC), which are involved in disulfide bond formation between the L and H chains; 2) a histidine-rich motif between positions 223-230 (HELxHxxH) associated with metalloprotease activity; and 3) a PYxGxAL motif (BoNT/A positions 635-645), located adjacent to a region identified as membrane spanning. There is a di-leucine motif (residues 427-428 of LC) only present in type A toxin gene, which may be critical for toxin trafficking (Steward et al., 2002). Consistent with previous findings, however, the C-terminal portion of the BoNT/A-Hall (AGN)/HC shows comparatively high sequence differences. The uniqueness and diversity of this putative receptor-binding region would support that the different toxin serotypes target different neuronal receptors, suggesting serotype-specific mechanisms of entry.

Different serotypes exhibit different duration of action (BoNT/A>BoNT/B>>BoNT/E) despite of that they have similar mechanism of action. A possible factor may be due to different target proteins (SNAP25, VAMP, synaptobrevin). However, this alone may not explain the different duration of action for BoNT/A and BoNT/E since they both target to the same protein, SNAP25, but have considerably different efficacy profiles. Another yet to be defined mechanism may be the different SNAP25 cleavage sites by BoNT/A or /E, which affect the half-lives of the cleavage products. Observations revealed that BoNT/A and /E have different half-life (Keller et al., 1999; Adler et al., 2001; Foran et al., 2003). Interestingly, neuronal signaling pathways are integrated with neurotoxin activity. Phosphorylation of BoNT/A, /B, /E, TeNT by neuronal protein kinases affects catalytic activity and stability of the toxins (Ferrer-Montiel et al., 1996; Gutierrez et al., 1997; Ferrer-Montiel et al., 1998). As such, different duration of action may be due to different modifications of toxins by neuronal enzymes, which lead to different compartmentalization of different toxins. Computer-assisted motif analysis reveals that toxins contain several potential sites for phosphorylation by casein kinase 11, protein kinase C, tyrosine kinases, glycogen synthase kinase 3, cGMP dependent protein kinase (PKG) that are well conserved (FIG. 3B). Note that the toxin also contains well-conserved N-glycosylation sites (FIG. 3B).

2.4. Functional Implications of Diversified Amino Acid Sequences

The difference in amino acid sequences may reflect the functional diversity such as pharmacology within different serotypes. Type A neurotoxin differs from type B neurotoxin in the safety margins in mice following intramuscular injection (Aoki, 2001 a; Aoki and Guyer, 2001). The safety margin for type A was 3-times as much as the experimental preparation of type B. One possible mechanism is that neurons at the murine neuromuscular junction internalize BoNT/A to a greater extent than BoNT/B (Black and Dolly, 1986). Thus, it is possible that in vivo more BoNT/B may remain outside the cell and, consequently, be more likely to escape from the muscle. The circulated BoNT/B may trigger a systemic effect, such as dry mouth. Cervical dystonia patients indeed showed a high incidence of dry mouth (22-44% with a 10,000 U dose of BoNT/B) (Lew et al., 1997; Brashear et al., 1999; Brin et al., 1999) while such an effect rarely observed with BoNT/A-Hall toxin complex (AGN) (refer to the product BOTOX®) treatment (Jankovic et al., 1990). As a structural support for this notion, the amino acid sequence in BoNT/A-Hall(AGN) differs from that in BoNT/B (Only 37.4% identical). The overall amino acid identity between BoNT/A-Hall (AGN) and other BoNT serotypes is low, around 40%. This may support a serotype-specific toxin trafficking, compartmentalization and action.

2.5. Phylogenetic Analysis

Traditionally, the genus Clostridium was classified on a basis of a few morphological, physiological and ultrastructural traits while phylogenetic data were missing. The genus Clostridium, defined phenotypically as containing Gram-positive, anaerobic rod-shaped, endospore-forming and neurotoxin-producing bacteria, consists of a phylogenetically incoherent species, in spite of being considered as descendants of a common ancestor emerged in the diversification of Gram-positive bacteria. Johnson and Francis pioneered the phylogenesis of 56 Clostridium species by determination of ribosomal ribonucleic acid (rRNA) homologies (Johnson and Francis, 1975). They defined the high degree of relatedness between C. botulinum (types A, B and F proteolytic) and between C. botulinum types C and D. In phylogenetic analysis, of the many macromolecules contained in a bacterial cell, only a few have been identified as suitable phylogenetic markers. Within one molecule, positions or regions that have different levels of conservation are informative for the analysis of different phylogenetic levels. The available data for the botulinum toxin complex genes show some degree of correlation in the branching patterns between BoNTs and the rRNA-defined relatedness (FIG. 4), such as type A and B may be related as well as type C and D may be related (Johnson and Francis, 1975). The symmetry of the dendogram in FIG. 4A is indicative of a large body of information that has been gathered allowing best alignments and creation of parsimony trees of relationship. FIG. 4C is however rather asymmetric with excessively lopsided branch lengths indicating that the dendogram would be a rather tentative relationship in lieu of better information about other Clostridium (or microbial) strains. With larger body of data regarding these strains the complex relationships of the strains might be more sensible from these proteins. At this point, we may argue that the placement of the “parsimonious” MRCA in dendogram 4 is probably incorrect with regard to relationship to the B and C groups versus A group of HA70 proteins. Therefore, the results of phylogenetic analysis using BoNTs, NTNHs and HA70, can not be superimposed onto the phylogenetic tree (FIG. 4), suggesting that they are diverse phylogenetically. Nonetheless, they may be functionally related since they contain some conserved regions such as catalytic region, translocation domain, and receptor binding domain in the neurotoxin protein. A comprehensive phylogenetic analysis of all available sequence data, combined with the classical polyphasic approach, will elucidate the relationships among different species and serotypes.

In summary, the DNA and predicted amino acid sequences of the neurotoxin protein complex for the C. botulinum type A-Hall (Allergan) strain are presented. This information may provide insight into the molecular basis of the interactions between toxic and nontoxic proteins in the macromolecular complex.

Various articles and patents have been cited here. The disclosures of these references are incorporated in their entirety herein by reference herein. The disclosures of the following references are also incorporated in their entirety herein by reference:
Adler, M., Keller, J. E., Sheridan, R. E. and Deshpande, S. S.: Persistence of botulinum neurotoxin A demonstrated by sequential administration of serotypes A and E in rat EDL muscle. Toxicon 39 (2001) 233-43.
Aoki, K. R.: A comparison of the safety margins of botulinum neurotoxin serotypes A, B, and F in mice. Toxicon 39 (2001a) 1815-20.
Aoki, K. R.: Pharmacology and immunology of botulinum toxin serotypes. J Neurol 248 Suppl 1 (2001b) 3-10.
Aoki, K. R. and Guyer, B.: Botulinum toxin type A and other botulinum toxin serotypes: a comparative review of biochemical and pharmacological actions. Eur J Neurol 8 Suppl 5 (2001) 21-9.
Bernard, C.: An introduction to the study of experimental medicine. Macmillan., New York., 1927.
Binder, W. J., Blitzer, A. and Brin, M. F.: Treatment of hyperfunctional lines of the face with botulinum toxin A. Dermatol Surg 24 (1998) 1198-205.
Binder, W. J., Brin, M. F., Blitzer, A. and Pogoda, J. M.: Botulinum toxin type A (BOTOX) for treatment of migraine. Dis Mon 48 (2002) 323-35.
Black, J. D and Dolly, J. O.: Interaction of 125I-labeled botulinum neurotoxins with nerve terminals. II. Autoradiographic evidence for its uptake into motor nerves by acceptor-mediated endocytosis. J Cell Biol 103 (1986) 535-44.
Brashear, A., Lew, M. F., Dykstra, D. D., Comella, C. L., Factor, S. A., Rodnitzky, R. L., Trosch, R., Singer, C., Brin, M. F., Murray, J. J., Wallace, J. D., Willmer-Hulme, A. and Koller, M.: Safety and efficacy of NeuroBloc (botulinum toxin type B) in type A-responsive cervical dystonia. Neurology 53 (1999) 1439-46.
Brin M F, H. M., Jankovic J: Scientific and therapeutic aspects of botulinum toxin. Lippincott Williams & Wilkins, Philadelphia, Baltimore, New York, London, Tokyo, 2002.
Brin, M. F., Lew, M. F., Adler, C. H., Comella, C. L., Factor, S. A., Jankovic, J., O'Brien, C., Murray, J. J., Wallace, J. D., Willmer-Hulme, A. and Koller, M.: Safety and efficacy of NeuroBloc (botulinum toxin type B) in type A-resistant cervical dystonia. Neurology 53 (1999) 1431-8.
Ferrer-Montiel, A. V., Canaves, J. M., DasGupta, B. R., Wilson, M .C. and Montal, M.: Tyrosine phosphorylation modulates the activity of clostridial neurotoxins. J Biol Chem 271 (1996) 18322-5.
Ferrer-Montiel, A. V., Gutierrez, L. M., Apland, J. P., Canaves, J. M., Gil, A., Viniegra, S., Biser, J. A., Adler, M. and Montal, M.: The 26-mer peptide released from SNAP-25 cleavage by botulinum neurotoxin E inhibits vesicle docking. FEBS Lett 435 (1998) 84-8.
Foran, P. G., Mohammed, N., Lisk, G. O., Nagwaney, S., Lawrence, G. W., Johnson, E., Smith, L., Aoki, K. R., Dolly, J. O.: Evaluation of the therapeutic usefulness of botulinum neurotoxin B, C1, E, and F compared with the long lasting type A. Basis for distinct durations of inhibition of exocytosis in central neurons. J. Biol Chem 278 (2003) 1363-71.
Gutierrez, L. M., Viniegra, S., Rueda, J., Ferrer-Montiel, A. V., Canaves, J. M. and Montal, M.: A peptide that mimics the C-terminal sequence of SNAP-25 inhibits secretory vesicle docking in chromaffin cells. J Biol Chem 272 (1997) 2634-9.
Henderson, I., Davis, T., Elmore, M. and Minton, N.: The genetic basis of toxin production in Clostridium botulinum and Clostridium tetani. In: Rood, J., McClane, B., Songer, J. and Titball, R. (Eds.), The Clostridia: Molecular biology and pathogenesis. Academic Press, San Diego, 1997, pp. 261-294.
Jankovic, J., Schwartz, K. and Donovan, D. T.: Botulinum toxin treatment of cranial-cervical dystonia, spasmodic dysphonia, other focal dystonias and hemifacial spasm. J Neurol Neurosurg Psychiatry 53 (1990) 633-9.
Johnson, E. A.: Clostridial toxins as therapeutic agents: benefits of nature's most toxic proteins. Annu Rev Microbiol 53 (1999) 551 -75.
Johnson, J. L. and Francis, B. S.: Taxonomy of the Clostridia: ribosomal ribonucleic acid homologies among the species. J Gen Microbiol 88 (1975) 229-44.
Keller, J. E., Neale, E. A., Oyler, G. and Adler, M.: Persistence of botulinum neurotoxin action in cultured spinal cord cells. FEBS Lett 456 (1999) 137-42.
Lacy, D. B., Tepp, W., Cohen, A. C., DasGupta, B. R. and Stevens, R. C.: Crystal structure of botulinum neurotoxin type A and implications for toxicity. Nat Struct Biol 5 (1998) 898-902.
Lew, M. F., Adornato, B. T., Duane, D. D., Dykstra, D. D., Factor, S. A., Massey, J. M., Brin, M. F., Jankovic, J., Rodnitzky, R. L., Singer, C., Swenson, M. R., Tarsy, D., Murray, J. J., Koller, M. and Wallace, J. D.: Botulinum toxin type B: a double-blind, placebo-controlled, safety and efficacy study in cervical dystonia. Neurology 49 (1997) 701-7.
Lin, W. J. and Johnson, E. A.: Transposon Tn916 mutagenesis in Clostridium botulinum. AppI Environ Microbiol 57 (1991) 2946-50.
Montecucco, C., Schiavo, G., Tugnoli, V. and de Grandis, D.: Botulinum neurotoxins: mechanism of action and therapeutic applications. Mol Med Today 2 (1996) 418-24.

Schantz, E. J. and Johnson, E. A .: Botulinum toxin: the story of its development for the treatment of human disease. Perspect Biol Med 40 (1997) 317-27.
Schiavo, G., Matteoli, M. and Montecucco, C.: Neurotoxins affecting neuroexocytosis. Physiol Rev 80 (2000) 717-66.
Schnider, P., Binder, M., Kittler, H., Birner, P., Starkel, D., Wolff, K. and Auff, E.: A randomized, double-blind, placebo-controlled trial of botulinum A toxin for severe axillary hyperhidrosis. Br J Dermatol 140 (1999) 677-80.
Simpson, L. L.: Identification of the characteristics that underlie botulinum toxin potency: implications for designing novel drugs. Biochimie 82 (2000) 943-53.
Steward, L. E., Fernandez-Salas, E., Ho, H., Sun, S. W., Ordas, J. V., Zhang, L., Herrington, T. M. and Aoki, K. R.: BoNT/A light chain and the dileucine motif: potential implications for light chain localization and neurotoxin duration of action. Naunyn-Schmiedeberg's Archives of Pharmacology. Supplement 2 to Volume 365 (2002).
Thompson, D. E., Brehm, J. K., Oultram, J. D., Swinfield, T. J., Shone, C. C., Atkinson, T., Melling, J. and Minton, N. P.: The complete amino acid sequence of the Clostridium botulinum type A neurotoxin, deduced by nucleotide sequence analysis of the encoding gene. Eur J Biochem 189 (1990) 73-81.
Botulinum toxin Hall A-hyper: GenBank Accession Number AF461540. Maniatis et al., Molecular Cloning: A Laboratory Manual, (Cold Spring Harbor Laboratory, Publisher, N.Y. (2d ed. 1989).

While this invention has been described with respect to various specific examples and embodiments, it is to be understood that the invention is not limited thereto and that it can be variously practiced with the scope of the following claims.

1. An isolated nucleic acid molecule comprising a nucleotide sequence (SEQ ID NO: 1) that encodes a botulinum toxin for type A-Hall (AGN) strain.

2. A vector comprising the molecule of claim 1.

3. A host cell or organism comprising the vector of claim 2.

4. A composition comprising the molecule of claim 1.

5. An isolated nucleic acid molecule comprising a nucleotide sequence (SEQ ID NO: 3) that encodes a type A-Hall (AGN) NTNH.

6. A vector comprising the molecule of claim 5.

7. A host cell or organism comprising the vector of claim 6.

8. A composition comprising the molecule of claim 5.

9. An isolated amino acid sequence comprising SEQ NO: 4 (type A-Hall (AGN) NTNH).

10. A composition comprising the isolated amino acid sequence of claim 9.

11. An isolated nucleic acid molecule comprising a nucleotide sequence (SEQ ID NO: 5) that encodes a Hall A/AGN HA70.

12. A vector comprising the molecule of claim 11.

13. A host cell or organism comprising the vector of claim 12.

14. A composition comprising the molecule of claim 11.

15. An isolated amino acid sequence comprising SEQ NO: 6 (type A-Hall (AGN) HA70).

16. A composition comprising the isolated amino acid sequence of claim 15.

17. An isolated nucleic acid molecule comprising a nucleotide sequence (SEQ ID NO: 7) that encodes a type A-Hall (AGN) HA34.

18. A vector comprising the molecule of claim 17.

19. A host cell or organism comprising the vector of claim 18.

20. A composition comprising the molecule of claim 17.

21. An isolated amino acid sequence comprising SEQ NO: 8 (type A-Hall (AGN) HA34).

22. A composition comprising the compound of claim 21.

23. An isolated nucleic acid molecule comprising a nucleotide sequence (SEQ ID NO: 9) that encodes a type A-Hall (AG N) HA17.

24. A vector comprising the molecule of claim 23.

25. A host cell or organism comprising the vector of claim 24.

26. A composition comprising the molecule of claim 23.

27. An isolated amino acid sequence comprising SEQ NO: 10 (type A-Hall (AGN) HA17).

28. A composition comprising the compound of claim 27.

29. An isolated nucleic acid molecule comprising a nucleotide sequence (SEQ ID NO: 11) that encodes a type A-Hall (AGN) botR/OrfX.

30. A vector comprising the molecule of claim 29.

31. A host cell or organism comprising the vector of claim 30.

32. A composition comprising the molecule of claim 29.

33. An isolated amino acid sequence comprising SEQ NO: 12 (type A-Hall (AGN) botR/OrfX).

34. A composition comprising the isolated amino acid sequence of claim 33.

35. A cell-free expression system comprising the vector of claim 2, 6, 12, 18, 24 or 30.

36. An expression vector system that simultaneously expresses one or more of the molecule of claim 1, 5, 11, 17, 23 or 29, wherein the expressed products are also assembled into a type A-Hall (AGN) toxin complex.


SEQUENCE LISTING (12 sequences)

SEQ ID NO: 1 - DNA, 3891 bp, C. Botulinum
atgccatttgttaataaacaatttaattataaagatcctgtaaatggtgttgatattgcttatataaaaattccaaatgcaggacaaatgcaaccagtaaaagcttttaaaattcataataaaatatgggttattccagaaagagatacatttacaaatcctgaagaaggagatttaaatccaccaccagaagcaaaacaagttccagtttcatattatgattcaacatatttaagtacagataatgaaaaagataattatttaaagggagttacaaaattatttgagagaatttattcaactgatcttggaagaatgttgttaacatcaatagtaaggggaataccattttggggtggaagtacaatagatacagaattaaaagttattgatactaattgtattaatgtgatacaaccagatggtagttatagatcagaagaacttaatctagtaataataggaccctcagctgatattatacagtttgaatgtaaaagctttggacatgaagttttgaatcttacgcgaaatggttatggctctactcaatacattagatttagcccagattttacatttggttttgaggagtcacttgaagttgatacaaatcctcttttaggtgcaggcaaatttgctacagatccagcagtaacattagcacatgaacttatacatgctggacatagattatatggaatagcaattaatccaaatagggtttttaaagtaaatactaatgcctattatgaaatgagtgggttagaagtaagctttgaggaacttagaacatttgggggacatgatgcaaagtttatagatagtttacaggaaaacgaatttcgtctatattattataataagtttaaagatatagcaagtacacttaataaagctaaatcaatagtaggtactactgcttcattacagtatatgaaaaatgtttttaaagagaaatatctcctatctgaagatacatctggaaaattttcggtagataaattaaaatttgataagttatacaaaatgttaacagagatttacacagaggataattttgttaagttttttaaagtacttaacagaaaaacatatttgaattttgataaagccgtatttaagataaatatagtacctaaggtaaattacacaatatatgatggatttaatttaagaaatacaaatttagcagcaaactttaatggtcaaaatacagaaattaataatatgaattttactaaactaaaaaattttactggattgtttgaattttataagttgctatgtgtaagagggataataacttctaaaactaaatcattagataaaggatacaataaggcattaaatgatttatgtatcaaagttaataattgggacttgttttttagtccttcagaagataattttactaatgatctaaataaaggagaagaaattacatctgatactaatatagaagcagcagaagaaaatattagtttagatttaatacaacaatattatttaacctttaattttgataatgaacctgaaaatatttcaatagaaaatctttcaagtgacattataggccaattagaacttatgcctaatatagaaagatttcctaatggaaaaaagtatgagttagataaatatactatgttccattatcttcgtgctcaagaatttgaacatggtaaatctaggattgctttaacaaattctgttaacgaagcattattaaatcctagtcgtgtttatacatttttttcttcagactatgtaaagaaagttaataaagctacggaggcagctatgtttttaggctgggtagaacaattagtatatgattttaccgatgaaactagcgaagtaagtactacggataaaattgcggatataactataattattccatatataggacctgctttaaatataggtaatatgttatataaagatgattttgtaggtgctttaatattttcaggagctgttattctgttagaatttataccagagattgcaatacctgtattaggtacttttgcacttgtatcatatattgcgaataaggttctaaccgttcaaacaatagataatgctttaagtaaaagaaatgaaaaatgggatgaggtctataaatatatagtaacaaattggttagcaaaggttaatacacagattgatctaataagaaaaaaaatgaaagaagctttagaaaatcaagcagaagcaacaaaggctataataaactatcagtataatcaatatactgaggaagagaaaaataatattaattttaatattgatgatttaagttcgaaacttaatgagtctataaataaagctatgattaatataaataaatttttgaatcaatgctctgtttcatatttaatgaattctatgatcccttatggtgttaaacggttagaagattttgatgctagtcttaaagatgcattattaaagtatatatatgataatagaggaactttaattggtcaagtagatagattaaaagataaagttaataatacacttagtacagatataccttttcagctttccaaatacgtagataatcaaagattattatctacatttactgaatatattaagaatattattaatacttctatattgaatttaagatatgaaagtaatcatttaatagacttatctaggtatgcatcaaaaataaatattggtagtaaagtaaattttgatccaatagataaaaatcaaattcaattatttaatttagaaagtagtaaaattgaggtaattttaaaaaatgctattgtatataatagtatgtatgaaaattttagtactagcttttggataagaattcctaagtattttaacagtataagtctaaataatgaatatacaataataaattgtatggaaaataattcaggatggaaagtatcacttaattatggtgaaataatctggactttacaggatactcaggaaataaaacaaagagtagtttttaaatacagtcaaatgattaatatatcagattatataaacagatggatttttgtaactatcactaataatagattaaataactctaaaatttatataaatggaagattaatagatcaaaaaccaatttcaaatttaggtaatattcatgctagtaataatataatgtttaaattagatggttgtagagatacacatagatatatttggataaaatattttaatctttttgataaggaattaaatgaaaaagaaatcaaagatttatatgataatcaatcaaattcaggtattttaaaagacttttggggtgattatttacaatatgataaaccatactatatgttaaatttatatgatccaaataaatatgtcgatgtaaataatgtaggtattagaggttatatgtatcttaaagggcctagaggtagcgtaatgactacaaacatttatttaaattcaagtttgtatagggggacaaaatttattataaaaaaatatgcttctggaaataaagataatattgttagaaataatgatcgtgtatatattaatgtagtagttaaaaataaagaatataggttagctactaatgcgtcacaggcaggcgtagaaaaaatactaagtgcattagaaatacctgatgtaggaaatctaagtcaagtagtagtaatgaagtcaaaaaatgatcaaggaataacaaataaatgcaaaatgaatttacaagataataatgggaatgatataggctttataggatttcatcagtttaataatatagctaaactagtagcaagtaattggtataatagacaaatagaaagatctagtaggactttgggttgctcatgggaatttattcctgtagatgatggatggggagaaaggccactgtaa

SEQ ID NO: 2 - protein, 1296 aa, C. Botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 3 - DNA, 3582 bp, C. Botulinum
atgaatataaatgacaacttaagtataaattccccggtagataataaaaatgttgtagtagttagagctagaaaaactgatacggtttttaaggcttttaaggttgctcccaatatttgggtggcgccagagagatattatggcgaatctttgagtatagatgaagaatataaagttgatgggggaatatatgattctaattttctttcacaagatagtgaaaaagataagttcttacaagccattattactttgttaaaaagaattaatagtactaacgctggggaaaagttattatctttgatttctacagctattccatttccttatggatatataggtggagggtattatgcacctaatatgattacttttggatcagcaccaaaatctaataaaaaattgaattctttaatttcaagtactattccatttccttatgcaggatatagagaaacaaattatctttcatctgaagataataaaagtttctatgcatctaatatagttatttttggtccaggagcaaacatagtagaaaacaatactgttttttataaaaaggaagatgcagaaaatggaatgggaacaatgactgaaatatggttccaaccatttctaacctataaatatgacgaattttatattgatcctgcaatagaattaataaaatgtttaataaaatctctttatttcttatatggtataaaacctagtgatgatttagttattccatatagattaagaagtgaattagagaatatagaatactcacaattgaatatagttgatttactagtatctggaggcattgatcctaaatttataaatacagatccatattggtttacagataattatttctcaaatgcaaaaaaagtgtttgaagatcataggaatatttatgaaacagaaattgaaggaaataatgccattggtaatgatataaaattgagattaaaacaaaagtttcgaatcaatatcaatgatatatgggaattaaatttaaattatttctctaaagagtttagcattatgatgccagatagatttaataatgcacttaaacatttttatagaaaacaatactacaaaatagattatccagaaaattatagtataaatggttttgttaatggtcaaattaatgctcaattatctttatcagatagaaatcaagatattataaataaacctgaagaaataattaatttattaaatggaaataatgtttcattaatgagaagtaatatttatggtgatggattaaaaagcactgtagatgatttttacagtaattataaaatcccatataatagagcctatgaatatcattttaataattcaaatgattcttctttagataatgttaacattggagtaatagacaatattccagagattatagatgtaaatccttataaggaaaattgtgataagttttcaccggtacagaaaattacaagtactagagaaattaatacaaatataccatggcctataaattatttacaagctcaaaatactaacaatgaaaaatttagtttatcctcagattttgtagaagtagtttcttctaaagataaatctttagtgtattctttcttatctaatgtaatgttttatttagattccataaaggataatagtcctattgatacagataaaaaatattatttatggttaagagagatttttagaaattattcttttgatattactgcaactcaagaaattaatactaattgtggtattaataaagtagtaacttggtttgggaaagcattaaatattttaaatacatctgattcttttgtagaagaatttcaaaatttaggggcaatttcacttattaataaaaaagaaaatttaagtatgccaataattgagagttatgaaatccctaacgatatgttaggattaccactaaatgatttaaatgaaaaattatttaacatatattctaaaaacacagcttattttaaaaaaatctactataatttcctagatcagtggtggacacaatattatagtcaatattttgatttaatttgtatggctaaaagatcagtgttagctcaagaaactttaataaaaagaataatacaaaaaaaattgagttatttaataggaaattctaatatatcatctgataacttagcattgatgaatcttacaacaacaaatacattaagagatatttcaaacgaatcacaaatagcaatgaataatgtagatagttttttaaataatgccgctatatgtgtttttgaaagtaatatatatcctaaatttatttcttttatggaacaatgtattaataatataaatattaagacaaaagaatttatacaaaaatgtactaatattaatgaagatgaaaaattacaattaattaaccagaatgtttttaatagcttagattttgaattcttaaatattcaaaatatgaaaagtttatttagttcagagacagcattacttataaaggaagaaacttggccttatgaactagtgttatatgcttttaaggaaccaggtaataatgttatcggagatgcatctggtaaaaatacatcaatagaatattctaaggacataggtttagtttatggaataaatagtgatgcattatatttaaatggatctaatcaaagtataagtttttctaatgatttctttgagaatggattaactaacagtttttcaatttatttttggttgagaaatttgggcaaagatactattaaatctaagttaataggtagtaaggaagataattgtggttgggaaatttattttcaagatactgggttggttttcaatatgatagattctaatggaaatgagaagaatatatatctatctgatgtttctaataatagttggcactatataactatatctgtagatcgtttaaaagaacaattattaatatttattgatgataatttagtggctaatgaaagtattaaggaaattttaaatatctattcaagtaatataatttctttattaagcgagaataatccaagttatattgagggattaactattttaaataaacccactacaagtcaggaagttttgagtaattattttgaagttctaaataattcatatataagagacagtaatgaagaacgattagaatacaataagacatatcaattatataattatgtattttcagataagcctatatgtgaagttaaacaaaataataatatatatttaacaattaataatacaaacaatttaaatctacaagcttctaaatttaaattattaagtataaatccaaataaacaatatgttcaaaaacttgatgaggtaataatttctgtattagataatatggaaaaatatatagatatatctgaagataatagattgcaactaatagataacaaaaataacgcaaagaagatgataattagtaatgatatatttatttccaattgtttaaccctatcttataacggtaaatatatatgtttatctatgaaagatgaaaaccataattggatgatatgtaataatgatatgtcaaagtatttgtatttatggtcatttaaataa

SEQ ID NO: 4 - protein, 1173 aa, C. Botulinum
MNINDNLSINSPVDNKNVVVVRARKTDTVFKAFKVAPNIWVAPERYYGESLSIDEEYKVDGGIYDSNFLSQDSEKDKFLQAIITLLKRINSTNAGEKLLSLISTAIPFPYGYIGGGYYAPNMITFGSAPKSNKKLNSLISSTIPFPYAGYRETNYLSSEDNKSFYASNIVIFGPGANIVENNTVFYKKEDAENGMGTMTEIWFQPFLTYKYDEFYIDPAIELIKCLIKSLYFLYGIKPSDNIVDLLVSGGIDPKFINTDPYWFTDNYFSNAKKVFEDHRNIYETEIEGNNAIGNDIKLRLKQKFRININDIWELNLNYFSKEFSIMMPDRFNNALKHFYRKQYYKIDYPENYSINGFVNGQINAQLSLSDRNQDIINKPEEIINLLNGNNVSLMRSNIYGDGLKSTVDDFYSNYKIPYNRAYEYHFNNSNDSSLDNVNIGVIDNIPEIIDVNPYKENCDKFSPVQKITSTREINTNIPWPINYLQAQNTNNEKFSLSSDFVEVVSSKDKSLVYSFLSNVMFYLDSIKDNSPIDTDKKYYLWLREIFRNYSFDITATQEINTNCGINKVVTWFGKALNILNTSDSFVEEFQNLGAISLINKKENLSMPIIESYEIPNDMLGLPLNDLNEKLFNIYSKNTAYFKKIYYNFLDQWWTQYYSQYFDLICMAKRSVLAQETLIKRIIQKKLSYLIGNSNISSDNLALMNLTTTNTLRDISNESQIAMNNVDSFLNNAAICVFESNIYPKFISFMEQCINNINIKTKEFIQKCTNINEDEKLQLINQNVFNSLDFEFLNIQNMKSLFSSETALLIKEETWPYELVLYAFKEPGNNVIGDASGKNTSIEYSKDIGLVYGINSDALYLNGSNQSISFSNDFFENGLTNSFSIYFWLRNLGKDTIKSKLIGSKEDNCGWEIYFQDTGLVFNMIDSNGNEKNIYLSDVSNNSWHYITISVDRLKEQLLIFIDDNLVANESIKEILNIYSSNIISLLSENNPSYIEGLTILNKPTTSQEVLSNYFEVLNNSYIRDSNEERLEYNKTYQLYNYVFSDKPICEVKQNNNIYLTINNTNNLNLQASKFKLLSINPNKQYVQKLDEVIISVLDNMEKYIDISEDNRLQLIDNKNNAKKMIISNDIFISNCLTLSYNGKYICLSMKDENHNWMICNNDMSKYLYLWSFK

SEQ ID NO: 5 - DNA, 1881 bp, C. Botulinum
atgaattcatctataaaaaaaatttataatgatatacaagaaaaagttataaactatagtgatactattgatttagctgatggtaattatgtagttagaagaggggatggatggatattatctagacaaaatcaaatattaggtggaagtgtaattagtaatggatcaacaggaatagttggggacctacgtgtaaatgataatgcgataccatattattatccaacaccatctttcaatgaagaatatataaaaaataatatacaaactgtatttactaactttactgaagctaatcaaattccaataggatttgaatttagtaaaaccgctccctcaaataaaaacttatatatgtatttacaatatacctacattagatatgaaataataaaagtcttacaacatgaaattatagaaagagcagttttatatgttccatctcttggatatgttaagtctatagaatttaatccaggggaaaaaataaataaagatttttactttctaactaatgataagtgcattttaaatgaacaattcctatataaaaaaattttagaaactactaaaaatataccaactaacaatatttttaattctaaagttagtagcacacaacgagtattgccttatagtaatgggctatatgttattaataagggtgatggatatataagaacaaatgataaagatttgataggtacattattaatcgaagcaggttcatcaggaagtattatacaacctcgattaagaaatacaactagaccattattcaccacaagtaatgatacaaaattctcacaacaatatactgaagaaagacttaaagacgctttcaatgtacaattatttaatacatcaacatcgttatttaaatttgtagaagaagctccttcagataaaaatatatgcataaaggcttataatacctatgaaaaatatgaattaatagactatcaaaatggaagtattgttaataaagctgagtattatcttccttccttaggatattgtgaagtaactaatgctccttcacctgaatctgaagtagttaaaatgcaagtggctgaagatggatttatacaaaatggtcccgaggaagaaattgtagtaggtgtcatagacccatctgaaaatatacaagaaataaatactgctatttcagataattacacatataacattccaggtattgtaaataataatccattttatatattatttacagtaaatactacaggaatttataaaattaatgctcaaaataatctaccatcattaaaaatatatgaagcgataggttctggtaatagaaatttccaatctgggaatttatgtgatgatgatattaaagcaataaattatattactgggtttgacagtcctaatgctaaaagttatttagttgttttgcttaataaggataaaaattactacattagagtaccacaaacttcttctaatatagaaaatcaaatacaattcaagagagaagaaggggatctccgaaatttaatgaattcttcagttaatataatagataatcttaattcaacaggtgcacattactatacaagacaaagccctgatgtccatgactatatttcatatgaatttacaatacctggtaactttaataataaagatacatctaacattaggctttatactagttataaccaaggaataggtactttatttagagtcactgaaactattgacggctataatttaattaatatacaacaaaatttacacctcttaaataataccaattcaatacgtttattaaatggtgcaatttatatattaaaagtagaagttacagaattaaataactataatataagattgcatatagatattactaattaa

SEQ ID NO: 6 - protein, 626 aa, C. Botulinum
MNSSIKKIYNDIQEKVINYSDTIDLADGNYVVRRGDGWILSRQNQILGGSVISNGSTGIVGDLRVNDNAIPYYYPTPSFNEEYIKNNIQTVFTNFTEANQIPIGFEFSKTAPSNKNLYMYLQYTYIRYEIIKVLQHEIIERAVLYVPSLGYVKSIEFNPGEKINKDFYFLTNDKCILNEQFLYKKILETTKNIPTNNIFNSKVSSTQRVLPYSNGLYVINKGDGYIRTNDKDLIGTLLIEAGSSGSIIQPRLRNTTRPLFTTSNDTKFSQQYTEERLKDAFNVQLFNTSTSLFKFVEEAPSDKNICIKAYNTYEKYELIDYQNGSIVNKAEYYLPSLGYCEVTNAPSPESEVVKMQVAEDGFIQNGPEEEIVVGVIDPSENIQEINTAISDNYTYNIPGIVNNNPFYILFTVNTTGIYKINAQNNLPSLKIYEAIGSGNRNFQSGNLCDDDIKAINYITGFDSPNAKSYLVVLLNKDKNYYIRVPQTSSNIENQIQFKREEGDLRNLMNSSVNIIDNLNSTGAHYYTRQSPDVHDYISYEFTIPGNFNNKDTSNIRLYTSYNQGIGTLFRVTETIDGYNLINIQQNLHLLNNTNSIRLLNGAIYILKVEVTELNNYNIRLHIDITN

SEQ ID NO: 7 - DNA, 882 bp, C. Botulinum
atggaacactattcagtaatccaaaattcattaaatgacaaaattgttaccatctcctgtaaggccgatactaatttatttttttatcaagttgccggtaacgttagcttatttcaacaaactagaaattaccttgaaagatggagacttatatatgattctaataaagctgcttataaaataaaaagtatggatatccataatactaatttagttttaacatggaatgcaccaacacataatatatcaacgcaacaagattcaaatgcagataatcaatattggttattattaaaagacattggtaacaattcatttattattgcaagttataaaaaccctaacttagtattatatgctgataccgtagctcgtaatttgaagcttagcacacttaataattcaaattatataaaatttatcatagaagattatataatatcagatcttaacaatttcacatgtaaaataagtccaatattagatcttaataaagttgtacaacaagtggatgtgacaaatctaaatgttaatttatatacttgggactatggtcgcaatcaaaaatggacaattagatataatgaagaaaaagcagcataccagttttttaatacaatactttcaaacggagttctaacatggattttttcaaatggtaatactgtaagggtttcttcttctaatgatcaaaataatgacgcccaatattggcttataaatcctgtttcagatactgatgaaacatatacaattactaatctacgcgatacaactaaagctctagatttatatggcggccaaacagcaaacggaactgctattcaagtatttaattatcatggagatgataatcagaaatggaatattcgtaacccataa

SEQ ID NO: 8 - protein, 293 aa, C. Botulinum
MEHYSVIQNSLNDKIVTISCKADTNLFFYQVAGNVSLFQQTRNYLERWRLIYDSNKAAYKIKSMDIHNTNLVLTWNAPTHNISTQQDSNADNQYWLLLKDIGNNSFIIASYKNPNLVLYADTVARNLKLSTLNNSNYIKFIIEDYIISDLNNFTCKISPILDLNKVVQQVDVTNLNVNLYTWDYGRNQKWTIRYNEEKAAYQFFNTILSNGVLTWIFSNGNTVRVSSSNDQNNDAQYWLINPVSDTDETYTITNLRDTTKALDLYGGQTANGTAIQVFNYHGDDNQKWNIRNP

SEQ ID NO: 9 - DNA, 441 bp, C. Botulinum
atgtcagttgaaagaacttttctacctaatggtaattacaatataaaatctatcttttctggttctttatatttaaatcctgtatcgaaatcattaacattttcaaatgaatcttctgcaaataatcaaaaatggaatgtagaatatatggctgaaaatagatgctttaaaatctctaatgtagcagaaccaaataagtatttaagttacgataactttggatttatttctttagattcattatccaatagatgctactggtttcctattaaaattgctgtaaatacttatattatgttaagtttaaataaagtgaatgaattagattatgcctgggacatttatgatactaatgaaaatattttaagccaaccactactcctattaccgaattttgatatatacaattcaaatcaaatgttcaaacttgaaaaaatataa

SEQ ID NO: 10 - protein, 146 aa, C. Botulinum
MSVERTFLPNGNYNIKSIFSGSLYLNPVSKSLTFSNESSANNQKWNVEYMAENRCFKISNVAEPNKYLSYDNFGFISLDSLSNRCYWFPIKIAVNTYIMLSLNKVNELDYAWDIYDTNENILSQPLLLLPNFDIYNSNQMFKLEKI

SEQ ID NO: 11 - DNA, 537 bp, C. Botulinum
atgaataagttgtttttacaaattaaaatgttaaaaaatgacaatagggagtttcaagaaatttttaagcattttgaaaaaactataaatatatttactagaaaatataatatatatgataattacaatgatattttgtaccatttatggtatacacttaaaaaagttgatttgagcaatttcaatacacaaaatgatttagagagatatattagtaggactttaaaaagatattgcttagatatttgcaataaaagaaagattgataagaaaataatatataattcagaaattgtagataagaaattaagcttaatagcaaatagttattcaagttatttagaatttgaatttaatgatttaatatccatattacctgatgatcaaaagaaaattatatatatgaaatttgttgaagatattaaggagatagatatagctaaaaaacttaatataagtcgtcaatctgtatataaaaataaaataatggctttagagagattagaacccatattgaaaaaattaattaatatgtag

SEQ ID NO: 12 - protein, 178 aa, C. Botulinum
MNKLFLQIKMLKNDNREFQEIFKHFEKTINIFTRKYNIYDNYNDILYHLWYTLKKVDLSNFNTQNDLERYISRTLKRYCLDICNKRKIDKKIIYNSEIVDKKLSLIANSYSSYLEFEFNDLISILPDDQKKIIYMKFVEDIKEIDIAKKLNISRQSVYKNKIMALERLEPILKKLINM